SEQUENCE LISTING (137 sequences)

SEQ ID NO: 1 - DNA, 1143 bp, Bacillus subtilis
  misc_feature (1)..(1140): Savinase-encoding sequence.
atgaagaaaccgttggggaaaattgtcgcaagcaccgcactactcatttctgttgcttttagttcatcgatcgcatcggctgctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcggtaccatggggaattagccgtgtgcaagccccagctgcccataaccgtggattgacaggttctggtgtaaaagttgctgtcctcgatacagggatatccactcatccagatctaaatattcgtggtggcgcaagctttgtaccaggggaaccgtcgactcaagatgggaatgggcatggcacgcatgtggccgggacgatcgctgctttaaacaattcgattggcgttcttggcgtagctcctagcgctgagctatacgctgttaaagtcctaggggcgagcggttcaggttcggtcagctcgattgcccaaggattggaatgggcagggaacaatggcatgcacgttgctaatttgagtttaggaagcccttcgccaagtgccacactcgagcaagctgttaatagcgcgacttctagaggcgttcttgttgtagcggcatctgggaattcaggtgcaggctcaatcagctatccggcgcgctatgcgaacgcaatggcagtcggagctactgatcaaaacaacaaccgcgctagcttttcacagtatggcgcaggccttgacattgtcgcacccggggtaaacgtgcagagcacatacccaggttcaacatatgccagcttaaacggtacatcgatggctactcctcatgttgcaggtgcggccgcccttgttaaacaaaagaacccatcttggtctaatgtacaaattcgaaatcatctaaagaatacggcaactagtttaggaagcacgaacttgtatggaagcggacttgttaacgcagaagcggcaacgcgttaa

SEQ ID NO: 2 - DNA, 31 bp, artificial sequence
  Table 1 Primer
gtagtacgtagcaccgtctaatggatttatg

SEQ ID NO: 3 - DNA, 31 bp, artificial sequence
  Table 1 Primer
gtgtcgacatgcaccgtctaatggatttatg

SEQ ID NO: 4 - DNA, 31 bp, artificial sequence
  Table 1 Primer
gtctatgtcagcaccgtctaatggatttatg

SEQ ID NO: 5 - DNA, 31 bp, artificial sequence
  Table 1 Primer
gttcatgtcagcaccgtctaatggatttatg

SEQ ID NO: 6 - DNA, 31 bp, artificial sequence
  Table 1 Primer
gtcgctctatgcaccgtctaatggatttatg

SEQ ID NO: 7 - DNA, 31 bp, artificial sequence
  Table 1 Primer
gtctacagacgcaccgtctaatggatttatg

SEQ ID NO: 8 - DNA, 31 bp, artificial sequence
  Table 1 Primer
gtgctcgtacgcaccgtctaatggatttatg

SEQ ID NO: 9 - DNA, 31 bp, artificial sequence
  Table 1 Primer
gttatgtgaggcaccgtctaatggatttatg

SEQ ID NO: 10 - DNA, 31 bp, artificial sequence
  Table 1 Primer
gtacatcgcagcaccgtctaatggatttatg

SEQ ID NO: 11 - DNA, 31 bp, artificial sequence
  Table 1 Primer
gtcagtcgtcgcaccgtctaatggatttatg

SEQ ID NO: 12 - DNA, 31 bp, artificial sequence
  Table 1 Primer
gttgacgagcgcaccgtctaatggatttatg

SEQ ID NO: 13 - DNA, 31 bp, artificial sequence
  Table 1 Primer
gtatatgctcgcaccgtctaatggatttatg

SEQ ID NO: 14 - DNA, 31 bp, artificial sequence
  Table 1 Primer
gtgacacagagcaccgtctaatggatttatg

SEQ ID NO: 15 - DNA, 31 bp, artificial sequence
  Table 1 Primer
gttactgatcgcaccgtctaatggatttatg

SEQ ID NO: 16 - DNA, 31 bp, artificial sequence
  Table 1 Primer
gtgtacgcgagcaccgtctaatggatttatg

SEQ ID NO: 17 - DNA, 31 bp, artificial sequence
  Table 1 Primer
gtcacgcgtggcaccgtctaatggatttatg

SEQ ID NO: 18 - DNA, 28 bp, artificial sequence
  Table 1 Primer
gtagtacgtacgattatggagcggattg

SEQ ID NO: 19 - DNA, 28 bp, artificial sequence
  Table 1 Primer
gtgtcgacatcgattatggagcggattg

SEQ ID NO: 20 - DNA, 28 bp, artificial sequence
  Table 1 Primer
gtctatgtcacgattatggagcggattg

SEQ ID NO: 21 - DNA, 28 bp, artificial sequence
  Table 1 Primer
gttcatgtcacgattatggagcggattg

SEQ ID NO: 22 - DNA, 28 bp, artificial sequence
  Table 1 Primer
gtcgctctatcgattatggagcggattg

SEQ ID NO: 23 - DNA, 28 bp, artificial sequence
  Table 1 Primer
gtctacagaccgattatggagcggattg

SEQ ID NO: 24 - DNA, 28 bp, artificial sequence
  Table 1 Primer
gtgctcgtaccgattatggagcggattg

SEQ ID NO: 25 - DNA, 28 bp, artificial sequence
  Table 1 Primer
gttatgtgagcgattatggagcggattg

SEQ ID NO: 26 - DNA, 28 bp, artificial sequence
  Table 1 Primer
gtacatcgcacgattatggagcggattg

SEQ ID NO: 27 - DNA, 28 bp, artificial sequence
  Table 1 Primer
gtcagtcgtccgattatggagcggattg

SEQ ID NO: 28 - DNA, 28 bp, artificial sequence
  Table 1 Primer
gttgacgagccgattatggagcggattg

SEQ ID NO: 29 - DNA, 28 bp, artificial sequence
  Table 1 Primer
gtatatgctccgattatggagcggattg

SEQ ID NO: 30 - DNA, 28 bp, artificial sequence
  Table 1 Primer
gtgacacagacgattatggagcggattg

SEQ ID NO: 31 - DNA, 28 bp, artificial sequence
  Table 1 Primer
gttactgatccgattatggagcggattg

SEQ ID NO: 32 - DNA, 28 bp, artificial sequence
  Table 1 Primer
gtgtacgcgacgattatggagcggattg

SEQ ID NO: 33 - DNA, 28 bp, artificial sequence
  Table 1 Primer
gtcacgcgtgcgattatggagcggattg

SEQ ID NO: 34 - DNA, 4378 bp, Bacillus subtilis
  misc_feature (1)..(4378): The xylR-xylA-xylB locus
cattgtaatcatgtccagaaaatgatcaatcacaatggaggacattcctaatgccggtgcattctgtcctaaggaagatggcaataattcatagctattgcctaattgggaataaacccttgatgatacttcacttctcattgaatttaaaaccataggatgcgattcaattatgctatttcttaaaattacggcttgtgggttgaaagtatttagaatattggtaaggcctattcctaaatagaatccaaaattttgtaatgcatttaaggttccgatatcattcagatgggcgaggtttatgatatcttgataggacagttttttctctttggtctgaagagattttaataaagccttctctgaagcatacaattcccagcatcctcggtttccgcaactgcatttaggaccattaaagtctattgtcatatgtcccatttctccagagaagccgcttactcctctatataaatgattgttgataataacaccgatccctattcctgtgctgatacttacgtaaataatgttatcgtgattttttgcagctccaaatagtttttctccatatgcgccagcatttgcctcattttcaataaaaacagacacattgtacttctcttgtatcgaagattttaagtcaatatctctccagttggagttcggagtgaaaacaattttttgatctttatcaatgagtccaggcacgcaaatacctataccaataaacccgtacggagattggggcatttgcgtaataaagtgatgaatcatatcaatcaaaatgtctttcgttatttctggagaattggattccaaatggcggtattgatcaagaacgattgttccttcaaggtctgttaaaatgccattaatataatccacaccaacatctattccaacggagtatcctgcctttttattaaaaacaagcatgacaggtcttcttccgccacttgattgtccttgacctatttcaaataccatactttctttcattaacgtgtttacctgtgatgagacagttgatttatttaatccagtcatttcagataattttgctcttgaaataggtgaatttttaaggatttcttttaataataacttttgatttacttttttgacaaaggtttgatcagcgatatccacttcatccactccatttgtttaatctttaaattaagtatcaacatagtacatagcgaatcttccctttattaaatctaatgtgttcataaaaaactaaaaaaaatattgaaaatactgacgaggttatataagatgaaaataagttagtttgtttaaacaacaaactaataggtgatgtacttactatatgaaataaaatgcatctgtatttgaatgaatttatttttaagggggaaatcacatggctcaatctcattccagttcaatcaactattttggaagcgcaaacaaagtggtttacgaagggaaagattcgactaatcctttagcatttaaatattataatcctcaagaagtaatcggcggaaaaacgctgaaagagcatttgcgattttctattgcctattggcatacatttactgctgatggtacagacgtttttggagcagctacgatgcaaagaccatgggatcactataaaggcatggatctagcgaagatgagagtagaagcagcatttgagatgtttgaaaaactagatgcaccattctttgcttttcatgaccgggatattgcaccagaaggcagtacgctaaaagagacaaaccaaaatttagatatgatcatgggcatgattaaagattacatgagaaatagcggcgttaagctattatggaatacagcaaacatgtttacgaatccccgtttcgtccatggtgccgcgacttcttgcaatgcagatgtgtttgcgtatgctgcagcacaagtgaaaaaagggttagaaacagcaaaagagcttggcgctgagaactatgtattttggggcggccgtgaaggatatgaaacattgttaaataccgatttaaaatttgagcttgatgatttggctagatttatgcatatggcagtggattatgcgaaggaaatcgggtacacagggcagtttttgattgagccaaaaccaaaagagccgaccgcccatcaatacgatacagatgcagcaacaaccattgcctttttgaagcaatatggcttagacaatcattttaaattaaatcttgaagccaatcatgccacattagccgggcatacattcgaacatgaattacgcatggcaagagtacatggtctgcttggctctgttgacgcaaaccagggtcatcctcttttaggctgggacacggatgaatttccgacggatttatattctacgacattagcaatgtacgaaatcctgcaaaatggcggccttggaagcggcggattaaactttgacgcgaaggtcagaagatcttctttcgagcctgatgatctaatatatgcccatattgcagggatggatgcatttgcaagaggattgaaagttgcccacaaattaatcgaagatcgtgtgtttgaagatgtgattcaacatcgttaccgcagctttactgaagggattggtcttgaaattatagaaggaagagctaatttccacacacttgagcaatatgcgctaaatcataaatcaattaaaaacgaatctggaagacaggagaaattaaaagcgatattgaaccaatacattttagaagtataacaggataagctccagatcctgctatcaataccaagtcactgaattacccgtcatgattcctttcctattgcttgttgttatgacgggtaacttctataattaggatttatttagagtgaatggttttttaaaagggcaaggagtgaaaaaatgaagtatgtcattggaatagatcttggaacgagtgctgttaaaaccattttagttaaccaaaacggcaaggtttgtgcagaaacgtccaaaaggtatccgctcatccaagagaaggcgggatatagtgagcaaaatcctgaagactgggttcagcaaacaattgaagcattggctgaattggtttctatatccaatgttcaagccaaggatattgacgggataagctattcgggacaaatgcatggattagtactgcttgaccaagatcgtcaggtgttacgtaatgcaattctttggaatgataccagaacaacgcctcaatgtataaggatgaccgagaaatttggcgatcatcttcttgacatcacaaaaaaccgtgttttagaagggtttacattacctaaaatgttatgggtaaaggaacatgaacctgaactttttaaaaaaactgctgtgtttttgcttccgaaagactacgtgcgattccgtatgaccggtgtcattcacaccgaatactccgatgcagcaggaactttacttttacatattactcgcaaggagtggagcaatgatatttgcaatcaaattggtatttctgcagatatttgtcctccgcttgttgaatctcatgattgtgtaggatcgctgcttccgcacgttgccgcgaagaccgggctattagaaaaaacaaaagtgtacgctgggggagcagataatgcttgcggcgctattggagcaggtatcctttcttccggaaaaacattatgcagtattgggacgtcaggggtcatactttcctacgaagaagaaaaagaaagagactttaaagggaaagtccacttttttaatcatggaaaaaaggattctttttatacgatgggcgtcacgctcgctgcaggatacagcttggactggtttaaaagaacgtttgcaccaaacgaatcgtttgagcaattattgcagggggtggaagctattccgataggagccaatggactgctatacactccttatttggttggtgaaagaacgccgcatgctgattcttctattcggggaagcttgatcggaatggatggagcccataatagaaagcattttttgagggcaataatggaaggtatcacattctctttacatgaatcaattgagctattccgcgaagcgggaaaatcagttcatactgttgtttctattggtgggggagctaaaaatgatacgtggctgcaaatgcaagctgatattttcaatacgagggtaattaagttagaaaatgaacaagggccagctatgggggctgcaatgctggctgcctttggaagcggttggtttgaatcacttgaagaatgtgcagagcagttcattcgtgaggctgctgcattttatccaaaggcgcaaaatgttcaaaaatataaaacactatttgatttgtataagaacatttacactcacacaaaggatctcaatacagctttgaagagctttcgaaaaaac

SEQ ID NO: 35 - DNA, 22 bp, artificial sequence
  Table 8. Primer sequences
cagaccatgtactcttgccatg

SEQ ID NO: 36 - DNA, 18 bp, artificial sequence
  Table 8. Primer sequences
gttctcagcgccaagctc

SEQ ID NO: 37 - DNA, 19 bp, artificial sequence
  Table 8. Primer sequences
ggtcttcttccgccacttg

SEQ ID NO: 38 - DNA, 19 bp, artificial sequence
  Table 8. Primer sequences
ccagttggagttcggagtg

SEQ ID NO: 39 - DNA, 22 bp, artificial sequence
  Table 8. Primer sequences
gaagcatacaattcccagcatc

SEQ ID NO: 40 - DNA, 19 bp, artificial sequence
  Table 8. Primer sequences
ctaatgccggtgcattctg

SEQ ID NO: 41 - DNA, 17 bp, artificial sequence
  Table 8. Primer sequences
gaagtcgcggcaccatg

SEQ ID NO: 42 - DNA, 29 bp, artificial sequence
  Table 9. Primer sequences
gtagacgtgcggtcttcttccgccacttg

SEQ ID NO: 43 - DNA, 29 bp, artificial sequence
  Table 9. Primer sequences
gttatgctgtggtcttcttccgccacttg

SEQ ID NO: 44 - DNA, 29 bp, artificial sequence
  Table 9. Primer sequences
gtacatgtcgggtcttcttccgccacttg

SEQ ID NO: 45 - DNA, 29 bp, artificial sequence
  Table 9. Primer sequences
gtctatgcgcggtcttcttccgccacttg

SEQ ID NO: 46 - DNA, 29 bp, artificial sequence
  Table 9. Primer sequences
gtatctatgcggtcttcttccgccacttg

SEQ ID NO: 47 - DNA, 29 bp, artificial sequence
  Table 9. Primer sequences
gtagatgctaggtcttcttccgccacttg

SEQ ID NO: 48 - DNA, 29 bp, artificial sequence
  Table 9. Primer sequences
gttagatagcggtcttcttccgccacttg

SEQ ID NO: 49 - DNA, 29 bp, artificial sequence
  Table 9. Primer sequences
gtatgtcagaggtcttcttccgccacttg

SEQ ID NO: 50 - DNA, 29 bp, artificial sequence
  Table 9. Primer sequences
gtgcgtctgtccagttggagttcggagtg

SEQ ID NO: 51 - DNA, 29 bp, artificial sequence
  Table 9. Primer sequences
gtctatgtgtccagttggagttcggagtg

SEQ ID NO: 52 - DNA, 29 bp, artificial sequence
  Table 9. Primer sequences
gtacgctgtcccagttggagttcggagtg

SEQ ID NO: 53 - DNA, 29 bp, artificial sequence
  Table 9. Primer sequences
gtatgtagcaccagttggagttcggagtg

SEQ ID NO: 54 - DNA, 32 bp, artificial sequence
  Table 9. Primer sequences
gtgcagatgcgaagcatacaattcccagcatc

SEQ ID NO: 55 - DNA, 32 bp, artificial sequence
  Table 9. Primer sequences
gtacgcgcgtgaagcatacaattcccagcatc

SEQ ID NO: 56 - DNA, 32 bp, artificial sequence
  Table 9. Primer sequences
gtctcgtatagaagcatacaattcccagcatc

SEQ ID NO: 57 - DNA, 32 bp, artificial sequence
  Table 9. Primer sequences
gttctactatgaagcatacaattcccagcatc

SEQ ID NO: 58 - DNA, 29 bp, artificial sequence
  Table 9. Primer sequences
gtatagatgcctaatgccggtgcattctg

SEQ ID NO: 59 - DNA, 29 bp, artificial sequence
  Table 9. Primer sequences
gtgatagagactaatgccggtgcattctg

SEQ ID NO: 60 - DNA, 29 bp, artificial sequence
  Table 9. Primer sequences
gtatagtcgactaatgccggtgcattctg

SEQ ID NO: 61 - DNA, 29 bp, artificial sequence
  Table 9. Primer sequences
gtgtcacgcgctaatgccggtgcattctg

SEQ ID NO: 62 - DNA, 29 bp, artificial sequence
  Table 9. Primer sequences
gtactgactgccagttggagttcggagtg

SEQ ID NO: 63 - DNA, 29 bp, artificial sequence
  Table 9. Primer sequences
gtagacgagtccagttggagttcggagtg

SEQ ID NO: 64 - DNA, 29 bp, artificial sequence
  Table 9. Primer sequences
gttcacacatccagttggagttcggagtg

SEQ ID NO: 65 - DNA, 29 bp, artificial sequence
  Table 9. Primer sequences
gtcgatactgccagttggagttcggagtg

SEQ ID NO: 66 - DNA, 28 bp, artificial sequence
  Table 9. Primer sequences
gtagacgtgcgttctcagcgccaagctc

SEQ ID NO: 67 - DNA, 28 bp, artificial sequence
  Table 9. Primer sequences
gttatgctgtgttctcagcgccaagctc

SEQ ID NO: 68 - DNA, 28 bp, artificial sequence
  Table 9. Primer sequences
gtacatgtcggttctcagcgccaagctc

SEQ ID NO: 69 - DNA, 28 bp, artificial sequence
  Table 9. Primer sequences
gtctatgcgcgttctcagcgccaagctc

SEQ ID NO: 70 - DNA, 32 bp, artificial sequence
  Table 9. Primer sequences
gtatctatgccagaccatgtactcttgccatg

SEQ ID NO: 71 - DNA, 32 bp, artificial sequence
  Table 9. Primer sequences
gtagatgctacagaccatgtactcttgccatg

SEQ ID NO: 72 - DNA, 32 bp, artificial sequence
  Table 9. Primer sequences
gttagatagccagaccatgtactcttgccatg

SEQ ID NO: 73 - DNA, 32 bp, artificial sequence
  Table 9. Primer sequences
gtatgtcagacagaccatgtactcttgccatg

SEQ ID NO: 74 - DNA, 32 bp, artificial sequence
  Table 9. Primer sequences
gtgcgtctgtcagaccatgtactcttgccatg

SEQ ID NO: 75 - DNA, 32 bp, artificial sequence
  Table 9. Primer sequences
gtctatgtgtcagaccatgtactcttgccatg

SEQ ID NO: 76 - DNA, 32 bp, artificial sequence
  Table 9. Primer sequences
gtacgctgtccagaccatgtactcttgccatg

SEQ ID NO: 77 - DNA, 32 bp, artificial sequence
  Table 9. Primer sequences
gtatgtagcacagaccatgtactcttgccatg

SEQ ID NO: 78 - DNA, 32 bp, artificial sequence
  Table 9. Primer sequences
gtgcagatgccagaccatgtactcttgccatg

SEQ ID NO: 79 - DNA, 32 bp, artificial sequence
  Table 9. Primer sequences
gtacgcgcgtcagaccatgtactcttgccatg

SEQ ID NO: 80 - DNA, 32 bp, artificial sequence
  Table 9. Primer sequences
gtctcgtatacagaccatgtactcttgccatg

SEQ ID NO: 81 - DNA, 32 bp, artificial sequence
  Table 9. Primer sequences
gttctactatcagaccatgtactcttgccatg

SEQ ID NO: 82 - DNA, 32 bp, artificial sequence
  Table 9. Primer sequences
gtatagatgccagaccatgtactcttgccatg

SEQ ID NO: 83 - DNA, 32 bp, artificial sequence
  Table 9. Primer sequences
gtgatagagacagaccatgtactcttgccatg

SEQ ID NO: 84 - DNA, 32 bp, artificial sequence
  Table 9. Primer sequences
gtatagtcgacagaccatgtactcttgccatg

SEQ ID NO: 85 - DNA, 32 bp, artificial sequence
  Table 9. Primer sequences
gtgtcacgcgcagaccatgtactcttgccatg

SEQ ID NO: 86 - DNA, 27 bp, artificial sequence
  Table 9. Primer sequences
gtactgactggaagtcgcggcaccatg

SEQ ID NO: 87 - DNA, 27 bp, artificial sequence
  Table 9. Primer sequences
gtagacgagtgaagtcgcggcaccatg

SEQ ID NO: 88 - DNA, 27 bp, artificial sequence
  Table 9. Primer sequences
gttcacacatgaagtcgcggcaccatg

SEQ ID NO: 89 - DNA, 27 bp, artificial sequence
  Table 9. Primer sequences
gtcgatactggaagtcgcggcaccatg

SEQ ID NO: 90 - DNA, 12 bp, artificial sequence
  Table 11. Primer sequences
agacgtgcggtc

SEQ ID NO: 91 - DNA, 12 bp, artificial sequence
  Table 11. Primer sequences
tatgctgtggtc

SEQ ID NO: 92 - DNA, 12 bp, artificial sequence
  Table 11. Primer sequences
acatgtcgggtc

SEQ ID NO: 93 - DNA, 12 bp, artificial sequence
  Table 11. Primer sequences
ctatgcgcggtc

SEQ ID NO: 94 - DNA, 12 bp, artificial sequence
  Table 11. Primer sequences
atctatgcggtc

SEQ ID NO: 95 - DNA, 12 bp, artificial sequence
  Table 11. Primer sequences
agatgctaggtc

SEQ ID NO: 96 - DNA, 12 bp, artificial sequence
  Table 11. Primer sequences
tagatagcggtc

SEQ ID NO: 97 - DNA, 12 bp, artificial sequence
  Table 11. Primer sequences
atgtcagaggtc

SEQ ID NO: 98 - DNA, 12 bp, artificial sequence
  Table 11. Primer sequences
gcgtctgtccag

SEQ ID NO: 99 - DNA, 12 bp, artificial sequence
  Table 11. Primer sequences
ctatgtgtccag

SEQ ID NO: 100 - DNA, 12 bp, artificial sequence
  Table 11. Primer sequences
acgctgtcccag

SEQ ID NO: 101 - DNA, 12 bp, artificial sequence
  Table 11. Primer sequences
atgtagcaccag

SEQ ID NO: 102 - DNA, 12 bp, artificial sequence
  Table 11. Primer sequences
gcagatgcgaag

SEQ ID NO: 103 - DNA, 12 bp, artificial sequence
  Table 11. Primer sequences
acgcgcgtgaag

SEQ ID NO: 104 - DNA, 12 bp, artificial sequence
  Table 11. Primer sequences
ctcgtatagaag

SEQ ID NO: 105 - DNA, 12 bp, artificial sequence
  Table 11. Primer sequences
tctactatgaag

SEQ ID NO: 106 - DNA, 12 bp, artificial sequence
  Table 11. Primer sequences
atagatgcctaa

SEQ ID NO: 107 - DNA, 12 bp, artificial sequence
  Table 11. Primer sequences
gatagagactaa

SEQ ID NO: 108 - DNA, 12 bp, artificial sequence
  Table 11. Primer sequences
atagtcgactaa

SEQ ID NO: 109 - DNA, 12 bp, artificial sequence
  Table 11. Primer sequences
gtcacgcgctaa

SEQ ID NO: 110 - DNA, 12 bp, artificial sequence
  Table 11. Primer sequences
actgactgccag

SEQ ID NO: 111 - DNA, 12 bp, artificial sequence
  Table 11. Primer sequences
agacgagtccag

SEQ ID NO: 112 - DNA, 12 bp, artificial sequence
  Table 11. Primer sequences
tcacacatccag

SEQ ID NO: 113 - DNA, 12 bp, artificial sequence
  Table 11. Primer sequences
cgatactgccag

SEQ ID NO: 114 - DNA, 12 bp, artificial sequence
  Table 11. Primer sequences
agacgtgcgttc

SEQ ID NO: 115 - DNA, 12 bp, artificial sequence
  Table 11. Primer sequences
tatgctgtgttc

SEQ ID NO: 116 - DNA, 12 bp, artificial sequence
  Table 11. Primer sequences
acatgtcggttc

SEQ ID NO: 117 - DNA, 12 bp, artificial sequence
  Table 11. Primer sequences
ctatgcgcgttc

SEQ ID NO: 118 - DNA, 12 bp, artificial sequence
  Table 11. Primer sequences
atctatgccaga

SEQ ID NO: 119 - DNA, 12 bp, artificial sequence
  Table 11. Primer sequences
agatgctacaga

SEQ ID NO: 120 - DNA, 12 bp, artificial sequence
  Table 11. Primer sequences
tagatagccaga

SEQ ID NO: 121 - DNA, 12 bp, artificial sequence
  Table 11. Primer sequences
atgtcagacaga

SEQ ID NO: 122 - DNA, 12 bp, artificial sequence
  Table 11. Primer sequences
gcgtctgtcaga

SEQ ID NO: 123 - DNA, 12 bp, artificial sequence
  Table 11. Primer sequences
ctatgtgtcaga

SEQ ID NO: 124 - DNA, 12 bp, artificial sequence
  Table 11. Primer sequences
acgctgtccaga

SEQ ID NO: 125 - DNA, 12 bp, artificial sequence
  Table 11. Primer sequences
atgtagcacaga

SEQ ID NO: 126 - DNA, 12 bp, artificial sequence
  Table 11. Primer sequences
gcagatgccaga

SEQ ID NO: 127 - DNA, 12 bp, artificial sequence
  Table 11. Primer sequences
acgcgcgtcaga

SEQ ID NO: 128 - DNA, 12 bp, artificial sequence
  Table 11. Primer sequences
ctcgtatacaga

SEQ ID NO: 129 - DNA, 12 bp, artificial sequence
  Table 11. Primer sequences
tctactatcaga

SEQ ID NO: 130 - DNA, 12 bp, artificial sequence
  Table 11. Primer sequences
atagatgccaga

SEQ ID NO: 131 - DNA, 12 bp, artificial sequence
  Table 11. Primer sequences
gatagagacaga

SEQ ID NO: 132 - DNA, 12 bp, artificial sequence
  Table 11. Primer sequences
atagtcgacaga

SEQ ID NO: 133 - DNA, 12 bp, artificial sequence
  Table 11. Primer sequences
gtcacgcgcaga

SEQ ID NO: 134 - DNA, 12 bp, artificial sequence
  Table 11. Primer sequences
actgactggaag

SEQ ID NO: 135 - DNA, 12 bp, artificial sequence
  Table 11. Primer sequences
agacgagtgaag

SEQ ID NO: 136 - DNA, 12 bp, artificial sequence
  Table 11. Primer sequences
tcacacatgaag

SEQ ID NO: 137 - DNA, 12 bp, artificial sequence
  Table 11. Primer sequences
cgatactggaag

(19) United States
(12) Patent Application Publication
(10) Pub. No.: US 2019/0048413 A1
(43) Pub. Date: Feb. 14, 2019

(54) IMPROVED NEXT-GENERATION SEQUENCING

(71) Applicant: NOVOZYMES A/S, Bagsvaerd (DK)

(72) Inventors: Marco Malten, Copenhagen NV (DK); Thomas Agersten Poulsen, Ballerup (DK); Preethi Ramaiya, Sunnyvale, CA (US); Michael Bargisen, Bagsvaerd (DK); Beth Nelson, Boulder Creek, CA (US)

(73) Assignee: NOVOZYMES A/S, Bagsvaerd (DK)

(21) Appl. No.: 16/079,141

(22) PCT Filed: Feb. 23, 2017
(86) PCT No.: PCT/US2017/019134

§ 371 Date: Aug. 23, 2018

Related U.S. Application Data
(60) Provisional application No. 62/298,899, filed on Feb. 23, 2016.

(51) Int. Cl. C12Q 1/6869; C12Q 1/6806; C12Q 1/6853; C12Q 1/686; C12Q 1/6876

(52) U.S. Cl. CPC C12Q 1/6869; C12Q 1/6806; C12Q 2600/166; C12Q 1/686; C12Q 1/6876; C12Q 1/6853

(57) ABSTRACT

The present invention enables the determination by NGS of the full nucleotide sequence of one or more polynucleotides of interest that are more than 800 bp in length after only one round of PCR amplification and subsequent fragmentation by adding at least one known oligonucleotide index to at least one end of each polynucleotide to produce elongated polynucleotides, wherein each known oligonucleotide index added to each polynucleotide is unique; fragmenting the elongated polynucleotides; sequencing the resulting fragments with paired forward and reverse reads using bridge amplification and next-generation sequencing; sorting the paired sequences based on the known oligonucleotide; and performing manual or in silico assembly of the sorted paired sequences, thereby providing the full coding sequence of each of the multitude of polynucleotides.

### REFERENCE TO SEQUENCE LISTING

This application contains a Sequence Listing in computer readable form. The computer readable form is incorporated herein by reference.

### FIELD OF THE INVENTION

The present invention enables the determination of the full nucleotide sequence of one or more polynucleotides of interest that are more than about 500 bp in length after only one round of PCR amplification, subsequent fragmentation and so-called next-generation sequencing.

### BACKGROUND OF THE INVENTION

The advent of the Illumina® DNA sequencing machines (Illumina® Inc. USA) enabled so-called “next-generation sequencing” (NGS) which is able to deliver continuous sequencing reads of about 300 nucleotides from a single read or about 500 nucleotides using both forward read (about 300 nucleotides) and reverse read (about 250 nucleotides) with 50-100 nucleotides' overlap to ensure proper merge of the two reads.

In order to sequence longer polynucleotides with NGS, the DNA is typically fragmented, the right size fragments (<500 nucleotides) are then isolated and an adaptor- or index-oligonucleotide is ligated onto the DNA fragments. The adaptors may carry a unique nucleotide sequence, an index, that can be used to differentiate between fragments in a mixed sample. The adaptors also have specific DNA sequences for annealing to the flow cell of the Illumina® NGS machines. Ligating these adaptors to the fragments requires the addition of enzymes and magnetic beads for DNA purification. The addition of the adaptors is somewhat time-consuming and it adds to the cost of the NGS process for longer sequences, of course. Novozymes routinely produces thousands of different new enzyme-encoding genes and needs the determine or verify the nucleotide sequences of those genes. Enzyme-encoding genes are usually between 800 and 3000 bp in length.

A sequencing process is needed that can provide the full sequences of thousands of enzyme-encoding genes or variants of a gene in the size-range of 800-3,000 bp quickly and at low cost.

### SUMMARY OF THE INVENTION

The present invention enables the determination by NGS of the full nucleotide sequence of one or more polynucleotides of interest that are more than 800 bp in length after only one round of PCR amplification and subsequent fragmentation. The PCR reaction may be performed directly on cultured cells comprising the polynucleotide(s) of interest, spores of such cells or other material comprising the polynucleotide(s) of interest.

A unique index oligonucleotide is introduced in the PCR amplification of each variant, e.g. on the forward primer of the PCR reaction. The PCR reactions are parallelized and the resulting PCR products are mixed (pooled) together. This mix of PCR products containing DNA from different variants is then fragmented, ideally using random fragmentation to get one randomly located cut per gene variant.

Next, the DNA fragments having a size above the NGS 500 bp sequencing limit are isolated, e.g. by cutting the fragments from an agarose gel. To the isolated fragments mix is then added sequencing adaptor oligonucleotides which may contain another index, so that this sample of interest can be multiplexed with other DNA sequencing samples on the same NGS run.

After the sequencing run, all paired sequence reads obtained for the samples are demultiplexed via the index sequence added during the first PCR. This is done so that, if the index is found on one read, the accordingly paired read is sorted to the same original gene variant (or position in a microtiter plate) as indicated by the unique indexes. When all reads obtained for the unique indexes are mapped/aligned against a reference sequence or are de novo assembled, a sequence longer than 800 bp can be obtained. See FIG. 1 for a schematic overview of the process with a single index primer and FIG. 2 for two index primers. The random fragmentation may be done, for example, by using a so-called fragmentase, or one or more restriction enzyme or by physical shearing. A fragmentase is an enzyme or enzyme mix used for random fragmentation of nucleic acids. It can consists of two enzymes, where one randomly generates nicks on doublestranded DNA and the other recognises the nicked site and cuts the opposite DNA strand across from the nick resulting a break of the double stranded DNA. Alternatively, a fragmentase system may use modification-dependent endonucleases requiring that modified bases are incorporated during a prior PCR step.

The fragmentation creates DNA pieces of different lengths, but only DNA pieces that still have the index at the start will anneal to the NGS flow cell. Hence, all reads obtained with the index always lie at the start of the PCR product. Each of these sorted reads also has a paired read from the NGS sequencing process. The paired read starts at the other end of the DNA piece. Due to the varying sizes of the DNA pieces after fragmentation its location varies between fragments.

The sequencing coverage near the start index will be always much higher than towards the other end of the PCR product where only the paired reads contribute. The only limit for this approach is the length of the DNA that can create paired reads. Illumina® Inc. currently specifies that 1 kb is the maximal DNA length which can be efficiently bridge-amplified on the flow cell of their machines. In the Illumina® process, bridge PCR or cluster PCR is used to amplify clonal sequences on the sequencing chip prior to sequencing. Single molecules of an adaptor-flanked DNA library are amplified on the chip with primers that densely coat the surface of the sequencing chip. This leads to closely tethered and locally contained clonal PCR product that can give a good signal during fluorescence based reversible termination sequencing. It is called bridge amplification, because after amplification of one strand, the DNA needs to bridge over so the other end of the DNA has contact with another primer on the chip to start the amplification of the reverse strand.

Using a forward index on the start of the PCR product and a reverse index at the end of the PCR product allows a doubling of the possible sequencing length, so that more than 1600 bp can be sequenced. The advantage of this method is the easy parallel preparation of a single individual PCR reaction for each variant, e.g. in 96 or 384 well PCR machines. Two indexed primers are used per variant. These primers are short, starting with two bases, an eight base long index sequence and an 18 to 22 base annealing region. The PCR reaction can be done in a very low volume (<5 microliter) because after pooling many individual samples, enough starting material is obtained for the subsequent fragmentation and fragment size selection. Here, e.g. for 96 or 384 samples only one further ligation of sequencing adaptors is needed.

Accordingly, in a first aspect the invention provides methods for determining the full coding sequences of a multitude of polynucleotides, said method comprising the steps of:
a) providing a multitude of polynucleotides,
b) adding at least one known oligonucleotide index to at least one end of each polynucleotide to produce elongated polynucleotides, wherein each known oligonucleotide index added to each polynucleotide is unique;
c) fragmenting the elongated polynucleotides;
d) sequencing the resulting fragments with paired forward and reverse reads using bridge amplification and next-generation sequencing;
e) sorting the paired forward and reverse sequence reads based on the at least one known oligonucleotide index; and
f) performing manual or in silico assembly of the sorted paired sequences, thereby providing the full coding sequence of each of the multitude of polynucleotides.

### BRIEF DESCRIPTION OF DRAWINGS

FIG. 1 shows a schematic of the sequencing principle of the invention with one index primer.

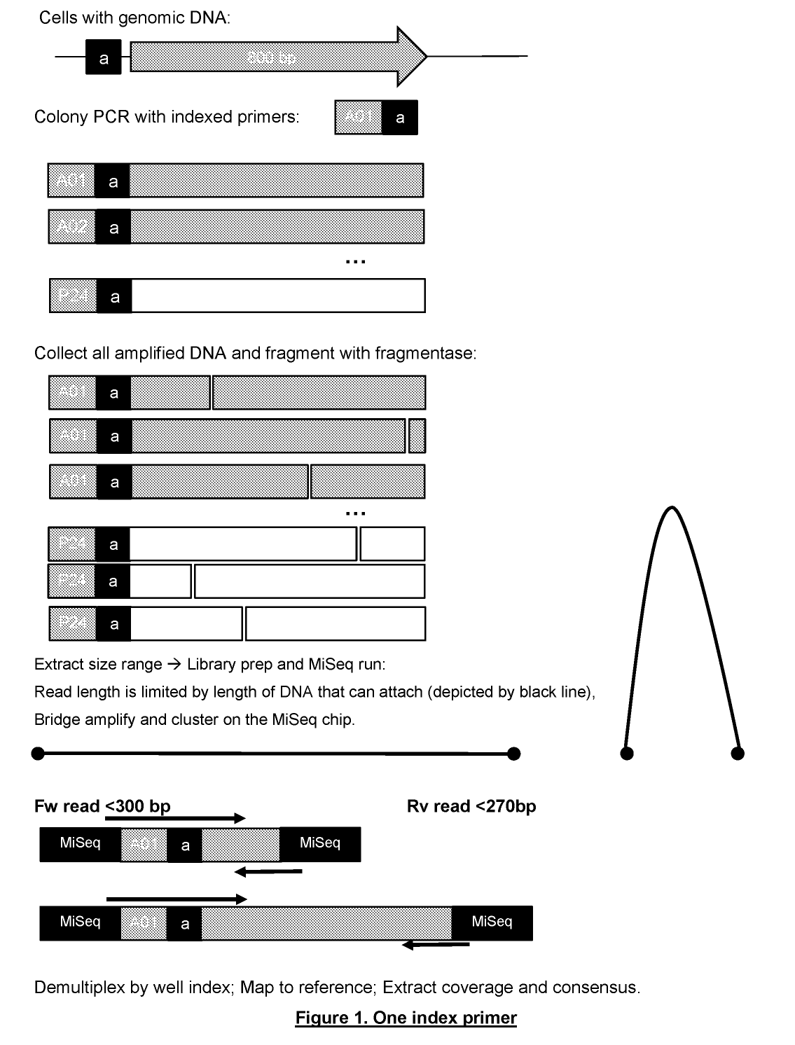

FIG. 2 shows a schematic of the sequencing principle of the invention with two index primers.

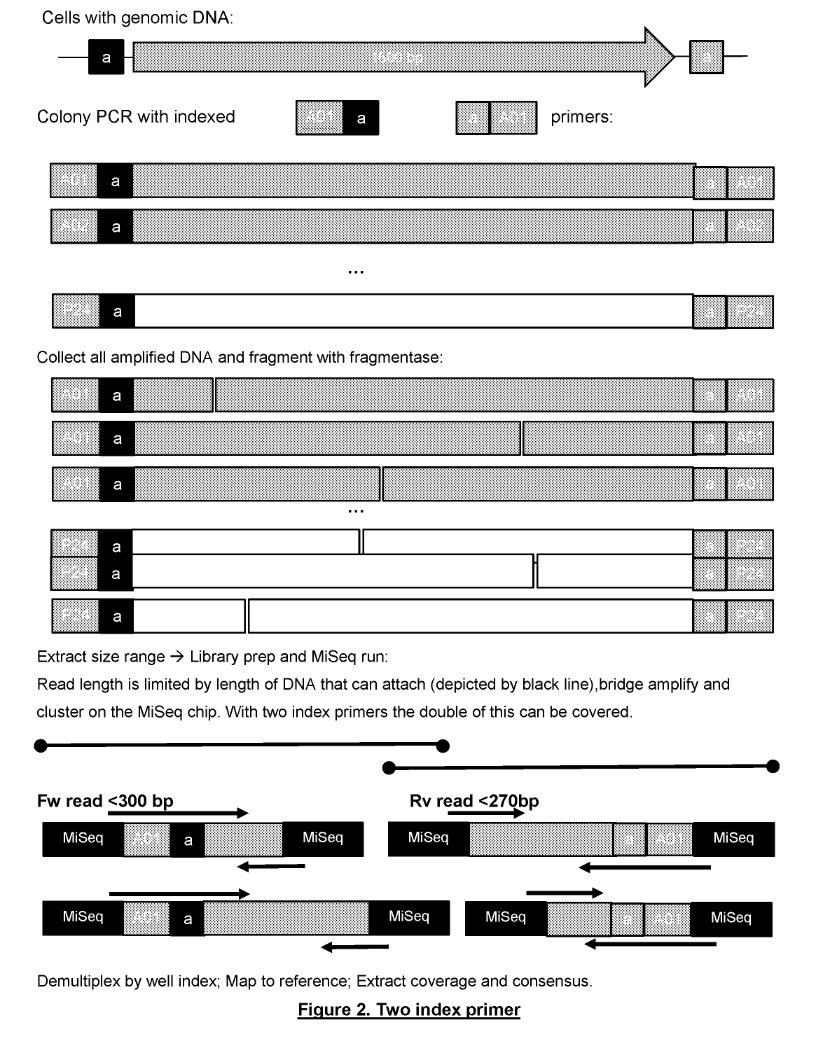

FIG. 3 shows photos of two agarose gel electrophoresis gels: The left gel shows 2 ug DNA after 10 or 12 min fragmentation. The right gel the same after cutting out agarose with fragmented DNA. Lane 1: DirectLoad™ Wide Range DNA Marker (Sigma Aldrich) showing DNA bands with 1500, 1400, 1000, 750, 500, 400, 300, 200, 100, 50 bp length. Lane 2: 10 min fragmentation; DNA in size range 800-1100 bp was extracted (called B2HDF). Lane 2: 10 min fragmentation; DNA in size range 500-1300 bp was extracted (called B2HDG). Lane 3: 10 min fragmentation; DNA in size range 100-1300 bp was extracted (B2HDJ). Lane 4: 12 min fragmentation; DNA in size range 800-1100 bp was extracted (called B2HDH). Lane 2: 12 min fragmentation; DNA in size range 500-1300 bp was extracted (called B2HDK). Lane 3: 12 min fragmentation; DNA in size range 100-1300 bp was extracted (B2HDM).

FIG. 4 shows graphs for each of the results from the 16 wells with different Savinase variants. Sequence coverage at the bottom is the coverage plotted at each 10th position for all 16 wells from position 7805 to 9005. The coding sequence of Savinase is from position 7867 to 9009. Coverage/total reads is the coverage divided by the total number of reads obtained for the sample in order to get a comparative value independent of the obtained reads. The position is the DNA. Note: Due to the limitations of grayscale pictures the different well lines are difficult to distinguish, but showing a distinct line for each visualizes the coverage range obtained across the 16 wells very well.

FIG. 5 shows photos of two agarose gel electrophoresis gels. 2 ug of different sized PCR amplicons were randomly fragmented with NEBNext dsDNA fragmentase (M0348S) and analyzed on an agarose gel. Lane 1 and 10 shows DirectLoad™ Wide Range DNA Marker (Sigma Aldrich) with a thick double band from 1400 and 1500 bp. Lane 3 shows A1-4, lane 4 A5-A8, lane 5 A9-A12, lane 6 A13-16, lane 7 A17-A20, lane 8 A21-24. The original sizes are 1040 bp for A1-4, 1361 bp for A5-8, 1650 bp for A9-12, 1940 bp for A13-16, 2257 bp for A17-20, 1247 bp for A21-24. The fragmentation pattern is seen as a smearing on the left picture. The right picture shows which size ranges were cut out for each PCR amplicon.

FIG. 6 shows the Mean Coverage of 4 PCR samples from primer set 1 to 4 for amplification of the same length PCR fragment. Mean coverage is plotted over reference position of the xylR-xylA-xylB genomic sequence.

FIG. 7 shows the Mean Coverage of 4 PCR samples with same length over reference position of the xylR-xylA-xylB genomic sequence.

### DEFINITIONS

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Next-generation sequencing: Next-generation sequencing utilizes a fundamentally different approach from the classic Sanger chain-termination method. It sequencing by DNA synthesis technology—tracking the addition of labeled nucleotides as the DNA chain is replicated—in a massively parallel fashion. Sequencing by synthesis (SBS) technology uses four fluorescently labeled nucleotides to sequence the tens of millions of dusters on an Illumina® flow cell surface in parallel. During each sequencing cycle, a single labeled deoxynucleoside triphosphate (dNTP) is added to the nucleic acid chain. The nucleotide label serves as a terminator for polymerization, so after each dNTP incorporation, the fluorescent dye is imaged to identity the base and then enzymatically cleaved to allow incorporation of the next nucleotide. Since all four reversible terminator-bound dNTPs (A, C, T, G) are present as single, separate molecules, natural competition minimizes incorporation bias. Base calls are made directly from signal intensity measurements during each cycle, which greatly reduces raw error rates compared to other technologies. The end result is highly accurate base-by-base sequencing that eliminates sequence-context specific errors, enabling robust base calling. A preferred device for next-generation sequencing employed in the first aspect of the invention is the commercially available Illumina® MiSeq machine (Illumina® Inc, USA).

Bridge-amplification: In contrast to the 454 and ABI methods which use a bead-based emulsion PCR to generate “polonies”, Illumina® utilizes a unique “bridged” amplification or bridge-amplification reaction that occurs on the surface of the flow cell. The flow cell surface is coated with single stranded oligonucleotides that correspond to the sequences of the adapters ligated during the sample preparation stage. Single-stranded, adapter-ligated fragments are bound to the surface of the flow cell exposed to reagents for polyermase-based extension. Priming occurs as the free/distal end of a ligated fragment “bridges” to a complementary oligo on the surface. Repeated denaturation and extension results in localized amplification of single molecules in millions of unique locations across the flow cell surface. This process occurs in what is referred to as Illumina's “cluster station”, an automated flow cell processor. Schematic images are also available on the web that explain the basic principle very well.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc.

Mature polypeptide coding sequence: The term “mature polypeptide coding sequence” means a polynucleotide that encodes a mature polypeptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

### DETAILED DESCRIPTION OF THE INVENTION

In a first aspect, the invention relates to methods for determining the full coding sequences of a multitude of polynucleotides, said methods comprising the steps of:
a) providing a multitude of polynucleotides,
b) adding at least one known oligonucleotide index to at least one end of each polynucleotide to produce elongated polynucleotides, wherein each known oligonucleotide index added to each polynucleotide is unique;
c) fragmenting the elongated polynucleotides;
d) sequencing the resulting fragments with paired forward and reverse reads using bridge amplification and next-generation sequencing;
e) sorting the paired forward and reverse sequence reads based on the at least one known oligonucleotide index; and
f) performing manual or in silico assembly of the sorted paired sequences, thereby providing the full coding sequence of each of the multitude of polynucleotides.

In a preferred embodiment, the multitude of polynucleotides of the first aspect of the invention comprise one or more promoters.

Alternatively, in another preferred embodiment of the first aspect, the multitude of polynucleotides encodes one or more polypeptide of interest or variants of one or more polypeptide of interest. Preferably, the one or more polypeptide of interest is one or more enzyme, preferably selected from the group of enzymes consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

In another preferred embodiment, the elongated polynucleotides in step (c) are randomly fragmented. Alternatively, the elongated polynucleotides in step (c) are fragmented by one or more endonuclease(s).

It is preferred that the bridge amplification and next-generation sequencing is performed in an Illumina® MiSeq sequencing machine (commercially available from Illumina® Inc, USA).

Preferably, the sorting step in the methods of the first aspect is done in silico. It is also preferred that the assembly is aided by alignment with a known reference nucleotide sequence.

### Sources of Polypeptides

The multitude of polynucleotides encoding polypeptides of interest in the first aspect of the invention may be obtained from microorganisms of any genus or they may be synthetic variants. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptides may be a bacterial polypeptides. For example, the polypeptides may be Gram-positive bacterial polypeptides such as Bacillus, Clostridium, Enterococcus, Geobacillus, Lactobacillus, Lactococcus, Oceanobacillus, Staphylococcus, Streptococcus, or Streptomyces polypeptide having [enzyme] activity, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma polypeptides.

Preferably, the polypeptides are Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, or Bacillus thuringiensis polypeptides.

Alternatively, the polypeptides may be Streptococcus equisimilis, Streptococcus pyogenes, Streptococcus uberis, or Streptococcus equi subsp. Zooepidemicus polypeptides, or Streptomyces achromogenes, Streptomyces avermitilis, Streptomyces coelicolor, Streptomyces griseus, or Streptomyces lividans polypeptides.

The polypeptides may be fungal. For example, the polypeptides may be yeast polypeptides such as Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, lrpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptides.

Preferably, the polypeptides are Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptides, or Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride polypeptides.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptides may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to polynucleotides encoding a polypeptides of interest, as described herein.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

The present invention is further described by the following examples that should not be construed as limiting the scope of the invention.

### EXAMPLES

### Example 1. Sequencing 16 Variants Using Dual Indexed Long PCRs with Fragmentase

This example describes how next-generation sequencing, here with an Illumina® Miseq machine (Illumina® Inc. USA), was used to obtain from one DNA library preparation the full 1257 bp length sequence in parallel for several variants of the same gene. The described principle is that a PCR product longer than the read length for the next-generation sequencer is coded with a unique index on both ends. Many of these individual PCR products are then mixed together. This mixed DNA is then fragmented to create different sized DNA pieces with only one index. To these DNA fragments, the next-generation sequencing adaptors are added and they are sequenced. The obtained paired reads are demultiplexed by the unique index, which means that the indexed read and the unindexed paired read are assigned to the original destination well of a microtiter plate, i.e. to a single variant gene. If the fragmentation is random, the unindexed read of the sequence read pair shall spread over the full length leading to a coverage that is very high in the ends and lower in the middle. The limitation for the maximal length of the sequence that can be covered with reads is the DNA fragment length that can be bridge amplified and form a cluster on the sequencing flow cell.

The present example was done with 16 known variants of a gene (SEQ ID NO:1) encoding the well-known protease Savinase which has a mature peptide length of 810 bp. Each Savinase variant gene was integrated in a Bacillus subtilis strain 168 and these Bacillus strains were cultivated over night, so that each well from A01 to A16 contains a distinct Savinase variant.

In the present example high concentrations of colony PCR products were prepared in order to get 2 microgram of DNA to work on. When 384 samples are used, 5 microliter reactions can be used to obtain enough DNA after mixing all 384 samples together.

For each of the 16 Savinase variants 100 microliter colony PCR reactions were run using 1 μl 33-fold diluted overnight culture as template, 25 μl ReddyMix (Thermo Scientific), 1 μl Fw primer (20 μM), 1 μl Rv primer (20 μM), 22 microl purified water. The PCR was run at 94° C. for 2 min 15 sec, followed by 35 cycles of 94° C. for 15 s, 58° C. for 25 s and 68° C. for 2 min. After the cycles a final elongation step was done at 68° C. for 4 min. For each of the 16 gene variants a different set of forward and reverse index primers was used. These index primers consists from the 5′ end of a GT, followed by a unique 8 nucleotide sequence, followed by an annealing sequence outside of the gene region that shall be sequenced.

TABLE 1
Primer sequences, wherein the first two nucleo-
tides are always G and T followed by an 8-nucleo-
tide index and the annealing nucleotides, for the
16 wells in the first row of a 96-well microplate

| | | | SEQ |
|---|---|---|---|
| | | | ID |
| Well | Direction | Sequence | NO |
| A01 | Forward | gtagtacgtagcaccgtctaatggatttatg | 2 |
| A02 | Forward | gtgtcgacatgcaccgtctaatggatttatg | 3 |
| A03 | Forward | gtctatgtcagcaccgtctaatggatttatg | 4 |
| A04 | Forward | gttcatgtcagcaccgtctaatggatttatg | 5 |
| A05 | Forward | gtcgctctatgcaccgtctaatggatttatg | 6 |
| A06 | Forward | gtctacagacgcaccgtctaatggatttatg | 7 |
| A07 | Forward | gtgctcgtacgcaccgtctaatggatttatg | 8 |
| A08 | Forward | gttatgtgaggcaccgtctaatggatttatg | 9 |
| A09 | Forward | gtacatcgcagcaccgtctaatggatttatg | 10 |
| A10 | Forward | gtcagtcgtcgcaccgtctaatggatttatg | 11 |
| A11 | Forward | gttgacgagcgcaccgtctaatggatttatg | 12 |
| A12 | Forward | gtatatgctcgcaccgtctaatggatttatg | 13 |
| A13 | Forward | gtgacacagagcaccgtctaatggatttatg | 14 |
| A14 | Forward | gttactgatcgcaccgtctaatggatttatg | 15 |
| A15 | Forward | gtgtacgcgagcaccgtctaatggatttatg | 16 |
| A16 | Forward | gtcacgcgtggcaccgtctaatggatttatg | 17 |
| A01 | Reverse | gtagtacgtacgattatggagcggattg | 18 |
| A02 | Reverse | gtgtcgacatcgattatggagcggattg | 19 |
| A03 | Reverse | gtctatgtcacgattatggagcggattg | 20 |
| A04 | Reverse | gttcatgtcacgattatggagcggattg | 21 |
| A05 | Reverse | gtcgctctatcgattatggagcggattg | 22 |
| A06 | Reverse | gtctacagaccgattatggagcggattg | 23 |
| A07 | Reverse | gtgctcgtaccgattatggagcggattg | 24 |
| A08 | Reverse | gttatgtgagcgattatggagcggattg | 25 |
| A09 | Reverse | gtacatcgcacgattatggagcggattg | 26 |
| A10 | Reverse | gtcagtcgtccgattatggagcggattg | 27 |
| A11 | Reverse | gttgacgagccgattatggagcggattg | 28 |
| A12 | Reverse | gtatatgctccgattatggagcggattg | 29 |
| A13 | Reverse | gtgacacagacgattatggagcggattg | 30 |
| A14 | Reverse | gttactgatccgattatggagcggattg | 31 |
| A15 | Reverse | gtgtacgcgacgattatggagcggattg | 32 |
| A16 | Reverse | gtcacgcgtgcgattatggagcggattg | 33 |

The resulting PCR products were checked for the correct size on agarose gels. The resulting PCR products were purified individually using a NucleoSpin Gel and PCR Clean-up kit with elution in 25 μl NE-buffer (5 mM Tris/HCl, pH 8.5). Concentration was measured by Qubit. After quantitation the 16 PCR products were pooled in equimolar concentration.

It was also shown that the PCR can be done with 2.5× diluted ReddyMix (Thermo Fisher Scientific) or with Phusion High-Fidelity DNA polymerase (Thermo Fisher Scientific).

TABLE 2
Concentrations measured of PCR products on Qubit
and amounts to make a large pool of 20 microgram,
which was later split to 2 microgram for each fragmentation

| Sample | Conc. (ng/μl) | μl for each 1250 ng |
|---|---|---|
| A01 | 298 | 4.19 |
| A02 | 368 | 3.40 |
| A03 | 390 | 3.21 |
| A04 | 352 | 3.55 |
| A05 | 406 | 3.08 |
| A06 | 236 | 5.30 |
| A07 | 406 | 3.08 |
| A08 | 172 | 7.27 |
| A09 | 308 | 4.06 |
| A10 | 504 | 2.48 |
| A11 | 542 | 2.31 |
| A12 | 218 | 5.73 |
| A13 | 372 | 3.36 |
| A14 | 145 | 8.62 |
| A15 | 254 | 4.92 |
| A16 | 348 | 3.59 |

6.8 μl equivalent to 2 μg of the pooled DNA sample, 2 μl 10× buffer, 9.2 I purified water and 2 μl NEB dsDNA Fragmentase were mixed and incubated either 10 or 12 minutes at 37° C. according to the description of the NEBNext dsDNA Fragmentase kit (M0348S).

Samples were analysed on an agarose gel showing some degradation of the DNA, visible by a smear below the band at 1.3 kb. For each sample a certain size range was cut out of the agarose and subsequently purified by NucleoSpin Gel Kit with elution in 25 μl NE-buffer. One digested DNA sample was not analysed by agarose gel electrophoresis, but was directly purified using a PCR Clean-up kit with elution in 25 μl NE-buffer. The DNA concentration of the obtained samples B1 to B7 each containing the indexed and fragmented DNA of wells A01 to A16 was measured, adjusted to 5 ng/μl and 20 μl were used to prepare libraries containing TruSeq dual indexes for Illumina® MiSeq sequencing.

TABLE 3
Overview of sample name, library ID, Sample, fragmentase incubation
time, purification method, cut out size, concentrations and total
number of reads, all amplified with with ReddyMix ™

| Sample | Library | | Time | | | Conc. | Total number |
|---|---|---|---|---|---|---|---|
| name | ID | Sample | (min) | Purification | Size | (ng/μl) | of reads |
| B1 | B2HDF | A01-A16 | 10 | gel + cut | 800-1100 | 8.33 | 517518 |
| B2 | B2HDG | A01-A16 | 10 | gel + cut | 500-1300 | 29.0 | 659142 |
| B3 | B2HDH | A01-A16 | 12 | gel + cut | 800-1100 | 5.54 | 544858 |
| B4 | B2HDJ | A01-A16 | 10 | gel + cut | 100-1300 | 28.7 | 1162636 |
| B5 | B2HDK | A01-A16 | 12 | gel + cut | 500-1100 | 11.1 | 665406 |
| B6 | B2HDM | A01-A16 | 12 | gel + cut | 100-1300 | 16.2 | 1294970 |
| B7 | B2HDN | A01-A16 | 12 | kit | all | 45.5 | 1229556 |

The KAPA Biosystems LT Library Prep Kit (KAPA Biosystems Cape Town, South Africa) was used to prepare the fragmented, size selected PCR products into Illumina® libraries. 100 ng starting material was used for each sample in a volume of 15 μl, using water to adjust the volume. The KAPA reagents were used to perform end repair, A-tailing, adaptor ligation. AMPureXP beads (Beckman Coulter) were used for all purification steps. Dual adaptors containing index sequences were obtained from Integrated DNA Technologies Coralville, Iowa. The adaptors contained different index sequences on each strand of DNA, allowing unique index sequence identification for both the forward and reverse reads of the same DNA molecule. The adaptors also facilitate the binding of the library molecules to the Illumina® flow cell and provides a universal sequence for library amplification. PCR reactions containing 15 μl prepared library, 25 μl KAPA HiFi HotStart ReadyMix, 2 μl amplification primers (50 μM) and 8 μl water were cycled at 98° C. 45 seconds once, 8× (98 C 15 seconds, 60 C 30 seconds, 72° C. 30 seconds) and 72° C. one minute. Purification of the PCR products using AMPure beads was performed following the KAPA Biosystems LT Library Prep Kit.

Quality control was performed on the resulting libraries to ensure accurate loading on the Illumina® MiSeq. Each library was analyzed on the Invitrogen Qubit to determine concentration. Then they were analyzed on the Fragment Analyzer (Advance Analytical, Ankeny, Iowa) using the Standard Sensitivity NGS Kit (DNF-473). Library molarity is calculated as: (ng/μl)*1500/average bp.

TABLE 4

| Designation | Library ID | Library ng/μl | Library Ave bp | Library nM |
|---|---|---|---|---|
| B2HDF | 2015_2142 | 5.7 | 1058 | 8.08 |
| B2HDG | 2015_2143 | 9.1 | 798 | 17.14 |
| B2HDH | 2015_2144 | 16.8 | 1106 | 22.78 |
| B2HDJ | 2015_2145 | 19.6 | 808 | 36.39 |
| B2HDK | 2015_2146 | 15.1 | 820 | 27.62 |
| B2HDM | 2015_2147 | 25.0 | 767 | 48.89 |
| B2HDN | 2015_2148 | 14.2 | 724 | 29.42 |

These libraries were pooled with other Miseq libraries. Each of the above libraries were calculated to be 2.3% of an Illumina® MiSeq run with a 600 bp V3 cartridge. The pool of libraries were denatured and diluted to 13 μM loading following the Illumina® standard protocol. The dual indexes in the adaptors were used to demultiplex the sequences and order these to the above samples and other samples on the same run.

The obtained paired read sequences of each sample B2HDF to B2HDN were each demultiplexed again using CLC Genomic benchmark software (CLC bio, a QIAGEN company, Aarhus, Denmark) sorting the paired reads by the 8 nucleotide long unique index plus the following 4 following nucleotides. First the forward (Fw) strand of the paired reads was searched, followed by searching the reverse (Rv) strand. For all samples 48 to 65% of the obtained sequences could be assigned to an index and with that to the microplate well (see table 5).

TABLE 5
CLC demultiplexing reports

| | | | Fw | Rv | | |
|---|---|---|---|---|---|---|
| | | | strand | strand | Total | |
| | | Run::Total | maps to | maps to | maps to | |
| | Sample | number of | A1 to | A1 to | A1 to | Av. reads |
| Designation | (R1/R2) | reads | A16 | A16 | A16 | per well |
| B2HDF | 2015_2142 | 517548 | 25% | 23% | 48% | 15526 |
| B2HDG | 2015_2143 | 659142 | 28% | 29% | 57% | 23482 |
| B2HDH | 2015_2144 | 544858 | 29% | 27% | 56% | 19070 |
| B2HDJ | 2015_2145 | 1162636 | 33% | 32% | 65% | 47232 |
| B2HDK | 2015_2146 | 665406 | 28% | 30% | 58% | 24121 |
| B2HDM | 2015_2147 | 1294970 | 29% | 32% | 61% | 49371 |
| B2HDN | 2015_2148 | 1229556 | 27% | 28% | 55% | 42266 |

The demultiplexing step was repeated using the flexbar program (Dodt et al. (2012), Biology 1(3), 895-905), as this is much easier and keeps well the pairing information. The following commands were used to first search the forward strand:

flexbar -r *B2HDF*_R1_001.fastq -p *B2HDF*_R2_001.fastq -b A16Fw+Rv.fasta -be LEFT -bt 0.5 -a adapters2015.fa -at 2 -t B2HDFR1

The A16Fw+RV.fasta contains a list of the 12 nucleotide long sequences used for searching and the adaptors2015.fa contains a list of adaptors sequences that could be contaminating. This flexbar command returns two files named_1 and _2 containing the paired forward and reverse reads. Next the reverse strand is searched by inverting the input of the forward and reverse reads:

flexbar -p *B2HDF*_R1_001.fastq -r *B2HDF*_R2_001.fastq -b A16Fw+Rv.fasta -be LEFT -bt 0.5 -a adapters2015.fa -at 2 -t B2HDFR2

This flexbar command returns two files named_1 and _2 containing the paired reverse and forward reads. Next the flexbar output files from both commands are concatenated resulting in a file named_1 and _2. Next these files were imported as Illumina® import of paired reads into the CLC Genomic Workbench (Quiagen). The reads of each well are trimmed at a limit of 0.01, mapped to the reference sequence, the mapping is locally realigned, the consensus extracted and probabilistic variant detection is run. A fasta format file (.fa) containing the consensus sequence, a mapping coverage table file (.tsv) containing information on nucleotide composition at each position is created. The coding sequence of the Savinase protease was used as reference (SEQ ID NO:1).

In summary, for each Bacillus clone expressing a different Savinase variant, a 1257 bp long colony PCR was made using a forward and a reverse primer starting with GT followed by a unique 8 nucleotide long index and a gene annealing region. This was done for 16 known Savinase variants based on SEQ ID 1. The PCR amplifications were done in large scale to obtain enough DNA to make several parallel tests. All 16 PCR products from different Savinase variants were then mixed equimolar and split up to be treated in different ways. 2 μg of DNA was fragmented with fragmentase for 10 or 12 minutes. Then DNA fragments of either 800-1100, 500-1100 (or 1300) or 200-1100 (or 1300) bp size ranges were cut out from an agarose gel and purified. The different samples were named as indicated in Table 6.

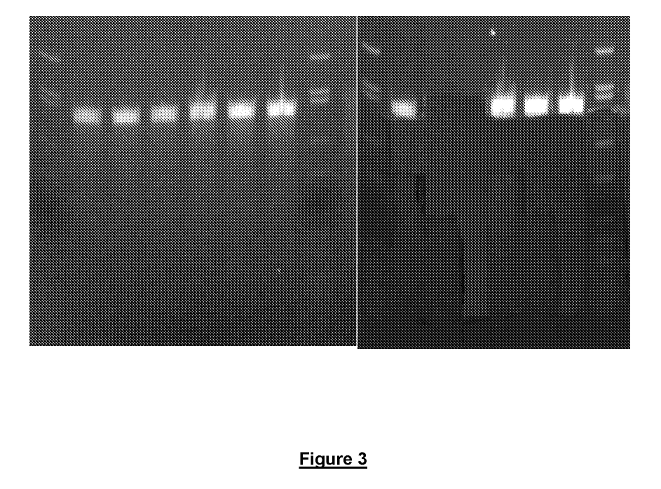

An agarose gel with the fragmented DNA before and after cutting out from the agarose gel is shown in FIG. 3. The purification of DNA from the agarose gel resulted in at least 5 ng/μl also if only 800-1100 bp were cut out. Hence, from a DNA starting material of only 2 μg, enough fragmented DNA can be obtained for the subsequent IIlumina® TrueSeq library preparation.

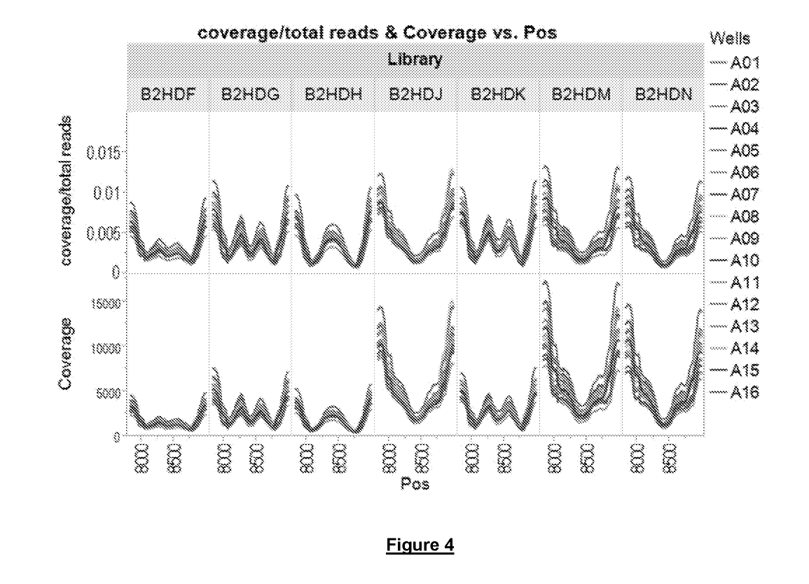

Phusion® High Fidelity (New England Biolabs) gave good results in the colony PCR. All 16 samples had more than 100-fold sequence coverage over the complete Savinase variant-encoding gene, with different profiles depending on the cut-out size range as seen in FIG. 4. If the cutoff is only at 100 bp, more sequences at the ends are obtained and a clear loss towards the middle is observed. However, the minimal obtained reads do not change a lot depending on the lower cutoff. One sample (B2HDN) was also only purified via a PCR purification kit, which allows all DNA above 100 bp to pass. The obtained results are very similar to the ones obtained with a lower 100 bp cutoff during extraction. Based on the minimal observed coverage it can be estimated that 2 million reads are needed for 384 different samples.

TABLE 6
A overview of the obtained sequence reads and an estimate of the reads needed for
384 samples. The columns describe: sample name and minutes of fragmentase treatment as
well as DNA fragment size range in bp; concentration after purification; total number of reads
from the IIlumina ® MiSeq sequencing; percentage of reads containing a valid index sequence;
average reads per well; lowest number of reads at any position of the mature peptide in any of
the 16 wells; reads estimated to be needed for 384 samples to obtain at least 100 reads on any
position; mean value of the coverage at all positions of the mature peptide of all the 16 wells.

| | | | Reads | | | | Mm |
|---|---|---|---|---|---|---|---|
| | | | map to | | | | (coverage)/ |
| | | Total | A1 to | Average | | | Total |
| Sample prep | conc. | number | A16 | reads per | Min | Mean | number of |
| (concat.) | (ng/ul) | of reads | (CLC) | well | (Coverage) | (Coverage) | reads |
| B2HDF 10 min | 8.33 | 517,518 | 48% | 15526 | 593 | 1,671 | 1.15E−3 |
| 800-1100 bp | | | | | | | |
| B2HDG 10 min | 29 | 659,142 | 57% | 23482 | 731 | 2,679 | 1.11E−3 |
| 500-1300 bp | | | | | | | |
| B2HDJ 10 min | 28.7 | 1,162,636 | 65% | 47232 | 1452 | 5,472 | 1.25E−3 |
| 100-1300 bp | | | | | | | |
| B2HDH 12 min | 5.54 | 544,858 | 56% | 19070 | 399 | 1,849 | 0.73E−3 |
| 800-1100 bp | | | | | | | |
| B2HDK 12 min | 11.1 | 665,406 | 58% | 24121 | 647 | 2,747 | 0.97E−3 |
| 500-1100 bp | | | | | | | |
| B2HDM 12 min | 16.2 | 1,294,970 | 61% | 49371 | 1555 | 5,828 | 1.20E−3 |
| 100-1300 bp | | | | | | | |
| B2HDN 12 min | 45.5 | 1,229,556 | 55% | 42266 | 960 | 4,969 | 0.78E−3 |
| kit all bp | | | | | | | |

Analyzing the consensus sequences showed that all 16 Savinase variants were sequenced correctly in all trials. Table 7 shows that setting the cutoffs at 800-1100 bp gave clearly more long reads, but the number of obtained indexed reads was reduced. This method of sequencing fragmentized long double indexed colony PCRs correctly determined the DNA sequences of 16 Savinase-variant encoding genes.

TABLE 7
Length distribution of sequence obtained between the Illumina ®
Miseq adaptors is shown dependent on the preparatation method and the
number of obtained reads. This data was visually extracted from
histograms supplied by the CLC genomic work bench of well A01.

| Number | | | | B2HDN |
|---|---|---|---|---|
| of reads | | | | No extraction, |
| obtained | B2DHF | B2DHG | B2DHJ | but purfication |
| with given | extract of | extract of | extract of | via PCR |
| length | 800-1100 bp | 500-1300 bp | 100-1300 bp | purification kit |
| >100 | 320-860 | 360-700 | 120-780 | 120-740 |
| >500 | | 510-590 | 260-660 | 260-600 |
| >1000 | | | 320-540 | 320-540 |

### Example 2. Determining Length of DNA Polynucleotides that can be Sequenced

This example shows that it is possible to sequence a 1.6 kb long DNA polynucleotide by fragmentation and double-indexing a colony PCR amplified piece of DNA according to the method of the invention using the Illumina® MiSeq NGS machine.

In this example each library was a combined sample of different lengths DNA amplified from the Bacillus genome (the xylR-xylA region; SEQ ID NO:34) in four reactions (length 1000, 1300, 1600, 1900, 2000). The 1000 bp long colony PCRs were prepared four times with A01 to A04 indexes, the 1300 bp with A05 to A08 indexes and so on. The quadruply amplified PCR fragments of each size were pooled and run on an agarose gel and DNA in the size-range from 800 bp to close below the original PCR sizes (1000, 1300, 1600, 1900, 2000 bp) were cut out and purified.

The different lengths were then pooled in an equimolar mix and an Illumina® MiSeq library was constructed by adding a dual index Illumina® adaptor. The loading of this library was 4.6% of the total MiSeq pool. After getting data for each of the libraries and demultiplexing to get sequences belonging to each of the 16 sequences, the data was analyzed to find how long the maximal sequencing length was.

TABLE 8
Primer sequences which were chosen as annealing
regions to amplify differrent length regions of
the xylR-xylA-xylB locus (SEQ ID NO: 34) from
Bacillus subtilis.

| | | | SEQ ID |
|---|---|---|---|
| Name | Oligo Sequence | Length | NO |
| anneal-CN | cagaccatgtactcttgccatg | 22 | 35 |
| anneal-CN-983 | gttctcagcgccaagctc | 18 | 36 |
| (w1300NC) | | | |
| anneal-NC-1300 | ggtcttcttccgccacttg | 19 | 37 |
| anneal-NC-1589 | ccagttggagttcggagtg | 19 | 38 |
| anneal-NC-1876 | gaagcatacaattcccagcatc | 22 | 39 |
| anneal-NC-2196 | ctaatgccggtgcattctg | 19 | 40 |
| anneal-CN-1191 | gaagtcgcggcaccatg | 17 | 41 |
| (w1583-NC) | | | |

The annealing sequences of table 8 were combined with the starting GT and an eight base long index sequences resulting in the used primers shown in Table 9, together with the index and the name of the used annealing region. The resulting fragment sizes are listed in Table 10 further down.

TABLE 9
Showing primer name; DNA sequence, wherein the first two nucleotides are always G
and T followed by an 8-nucleotide linker before the annealing nucleotides; fragment
size between primers; index sequence; annealing name; SEQ ID NO.

| | | | annealing | SEQ |
|---|---|---|---|---|
| Name | Sequence incl. index | size | name | ID NO |
| 0120-NC | gtagacgtgcggtcttcttccgccacttg | 983 | anneal-NC-1300 | 42 |
| 0121-NC | gttatgctgtggtcttcttccgccacttg | 983 | anneal-NC-1300 | 43 |
| 0122-NC | gtacatgtcgggtcttcttccgccacttg | 983 | anneal-NC-1300 | 44 |
| 0123-NC | gtctatgcgcggtcttcttccgccacttg | 983 | anneal-NC-1300 | 45 |
| 0124-NC | gtatctatgcggtcttcttccgccacttg | 1300 | anneal-NC-1300 | 46 |
| 0125-NC | gtagatgctaggtcttcttccgccacttg | 1300 | anneal-NC-1300 | 47 |
| 0126-NC | gttagatagcggtcttcttccgccacttg | 1300 | anneal-NC-1300 | 48 |
| 0127-NC | gtatgtcagaggtcttcttccgccacttg | 1300 | anneal-NC-1300 | 49 |
| 0128-NC | gtgcgtctgtccagttggagttcggagtg | 1589 | anneal-NC-1589 | 50 |
| 0129-NC | gtctatgtgtccagttggagttcggagtg | 1589 | anneal-NC-1589 | 51 |
| 0130-NC | gtacgctgtcccagttggagttcggagtg | 1589 | anneal-NC-1589 | 52 |
| 0131-NC | gtatgtagcaccagttggagttcggagtg | 1589 | anneal-NC-1589 | 53 |
| 0132-NC | gtgcagatgcgaagcatacaattcccagcatc | 1876 | anneal-NC-1876 | 54 |
| 0133-NC | gtacgcgcgtgaagcatacaattcccagcatc | 1876 | anneal-NC-1876 | 55 |
| 0134-NC | gtctcgtatagaagcatacaattcccagcatc | 1876 | anneal-NC-1876 | 56 |
| 0135-NC | gttctactatgaagcatacaattcccagcatc | 1876 | anneal-NC-1876 | 57 |
| 0136-NC | gtatagatgcctaatgccggtgcattctg | 2196 | anneal-NC-2196 | 58 |
| 0137-NC | gtgatagagactaatgccggtgcattctg | 2196 | anneal-NC-2196 | 59 |
| 0138-NC | gtatagtcgactaatgccggtgcattctg | 2196 | anneal-NC-2196 | 60 |
| 0139-NC | gtgtcacgcgctaatgccggtgcattctg | 2196 | anneal-NC-2196 | 61 |
| 0140-NC | gtactgactgccagttggagttcggagtg | 1191 | anneal-NC-1589 | 62 |
| 0141-NC | gtagacgagtccagttggagttcggagtg | 1191 | anneal-NC-1589 | 63 |
| 0142-NC | gttcacacatccagttggagttcggagtg | 1191 | anneal-NC-1589 | 64 |
| 0143-NC | gtcgatactgccagttggagttcggagtg | 1191 | anneal-NC-1589 | 65 |
| 0144-CN | gtagacgtgcgttctcagcgccaagctc | 983 | anneal-CN-983 | 66 |
| | | | (w1300NC) | |
| 0145-CN | gttatgctgtgttctcagcgccaagctc | 983 | anneal-CN-983 | 67 |
| | | | (w1300NC) | |
| 0146-CN | gtacatgtcggttctcagcgccaagctc | 983 | anneal-CN-983 | 68 |
| | | | (w1300NC) | |
| 0147-CN | gtctatgcgcgttctcagcgccaagctc | 983 | anneal-CN-983 | 69 |
| | | | (w1300NC) | |
| 0148-CN | gtatctatgccagaccatgtactcttgccatg | 1300 | anneal-CN | 70 |
| 0149-CN | gtagatgctacagaccatgtactcttgccatg | 1300 | anneal-CN | 71 |
| 0150-CN | gttagatagccagaccatgtactcttgccatg | 1300 | anneal-CN | 72 |
| 0151-CN | gtatgtcagacagaccatgtactcttgccatg | 1300 | anneal-CN | 73 |
| 0152-CN | gtgcgtctgtcagaccatgtactcttgccatg | 1589 | anneal-CN | 74 |
| 0153-CN | gtctatgtgtcagaccatgtactcttgccatg | 1589 | anneal-CN | 75 |
| 0154-CN | gtacgctgtccagaccatgtactcttgccatg | 1589 | anneal-CN | 76 |
| 0155-CN | gtatgtagcacagaccatgtactcttgccatg | 1589 | anneal-CN | 77 |
| 0156-CN | gtgcagatgccagaccatgtactcttgccatg | 1876 | anneal-CN | 78 |
| 0157-CN | gtacgcgcgtcagaccatgtactcttgccatg | 1876 | anneal-CN | 79 |
| 0158-CN | gtctcgtatacagaccatgtactcttgccatg | 1876 | anneal-CN | 80 |
| 0159-CN | gttctactatcagaccatgtactcttgccatg | 1876 | anneal-CN | 81 |
| 0160-CN | gtatagatgccagaccatgtactcttgccatg | 2196 | anneal-CN | 82 |
| 0161-CN | gtgatagagacagaccatgtactcttgccatg | 2196 | anneal-CN | 83 |
| 0162-CN | gtatagtcgacagaccatgtactcttgccatg | 2196 | anneal-CN | 84 |
| 0163-CN | gtgtcacgcgcagaccatgtactcttgccatg | 2196 | anneal-CN | 85 |
| 0164-CN | gtactgactggaagtcgcggcaccatg | 1191 | anneal-CN-1191 | 86 |
| | | | (w1583-NC) | |
| 0165-CN | gtagacgagtgaagtcgcggcaccatg | 1191 | anneal-CN-1191 | 87 |
| | | | (w1583-NC) | |
| 0166-CN | gttcacacatgaagtcgcggcaccatg | 1191 | anneal-CN-1191 | 88 |
| | | | (w1583-NC) | |
| 0167-CN | gtcgatactggaagtcgcggcaccatg | 1191 | anneal-CN-1191 | 89 |
| | | | (w1583-NC) | |

TABLE 10

| | | | | Fragment |
|---|---|---|---|---|
| | | | Fragment | size between |
| Wells | NC primer | CN primer | total size | primers |
| A01-A04 | 0120-0123 | 0144-0147 | 1040 | 983 |
| A05-A08 | 0124-0127 | 0148-0151 | 1361 | 1300 |
| A09-A12 | 0128-0131 | 0152-0155 | 1650 | 1589 |
| A13-A16 | 0132-0135 | 0156-0159 | 1940 | 1876 |
| A17-A20 | 0136-0139 | 0160-0163 | 2257 | 2196 |
| A21-A24 | 0140-0143 | 0164-0167 | 1247 | 1191 |

Bacillus subtilis 168 strain was used as a template for the PCR amplification of the xylR-xylA-xylB locus (SEQ ID NO:2) according to the following PCR protocol:

| MasterMix for one 384-well plate | Program used |
|---|---|
| 1320 μl HF-buffer | Cycle: |
| 30 μl Phusion | 98° C. for 15 s; |
| 211.3 μl dNTP (2.5 mM each) | 58° C. for 30 s; and |
| 2340 μl H₂O | 72° C. for 2 min. |
| Total = 3960 μl | 30 times |
| 96 μl of this master-mix was pipetted in | 72° C. for 5 min |
| the PCR-plate. | Store at 10° C. |
| 32 μl of Bacillus 168 DNA (40 ng/μl) and | |
| 32 μl of a 2 μM dilution of the combined | |
| Row + Plate primers were added; 1 μl | |
| 100 μM of each primer in 48 μl H₂O. | |

Total volume 40 μl per well/PCR.

An agarose gel of small aliquots showed that PCR amplicons with four replicates of each size were present and had the correct length. After mixing the four replicates, the PCR amplicons were purified by a NucleoSpin Gel and PCR Clean-up kit and eluted in 65 μl NE-buffer. Then concentration was measured on a Qubit giving 188 ng/μl for A1-4, 200 ng/μl for A5-8, 212 ng/μl for A9-12, 150 ng/μl for A13-16, 163 ng/μl for A17-20, and 163 ng/μl for A21-24.

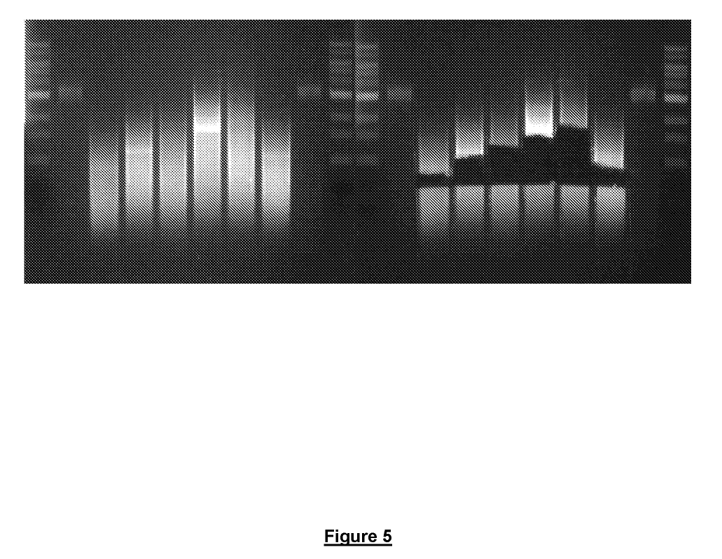

2 μg of each were then digested for 11 min at 37° C. with 2 μl NEBNext dsDNA Fragmentase (M0348S) in a total of 20 μl. After fragmentation 5 μl 0.5M, pH 8.0 was added to stop the reaction. 2 digested samples were pooled and different size bands separated by agarose gel electrophoreses as shown in FIG. 5.

After fragmentation a smear of lower sized fragmentation products was seen below the original size band. For each sample (A1-4, A5-8, A9-12, A13-16, A17-20, A21-24) the bands in the size range from 800 bp to just below the original PCR amplicon length band were cut out from the gel (as shown in FIG. 5), purified on a NucleoSpin Gel and PCR Clean-up kit and eluted in 30 ul NE-buffer.

The Qubit measured concentrations were 4.03 ng/μl for A1-4, 18.1 for A5-8, 23.3 ng/μl for A9-12, 37.4 ng/μl for A13-A16, 40.8 ng/μl for A17-A20 and 13.6 ng/μl for A21-A24. Of each 120 ng were then mixed together and 63.5 μl NE-buffer were added to fill up to 120 μl resulting in a concentration of 6 ng/μl. This way fragments from different length starting DNA were mixed into one sample. This ensures that all were treated the same way and a direct comparision between the sequencing length for the different size length was obtained.

These fragments were constructed into an Illumina® library using the commercially available KAPA HyperPlus Library Preparation Kit (Kapa Biosystems), but omitting the fragmentation step. No size selection was performed and the post-adaptor ligation clean-up was performed twice with 0.8× AMPure beads. PCR amplification containing 20 μl prepared library, 25 μl KAPA HiFi HotStart ReadyMix, 5 μl amplification primers (10× supplied by KAPA) was cycled at 98° C. 3 minutes once, 10× (98° C. 1 minute 20 seconds, 60° C. 30 seconds, 72° C. 30 seconds) and 72° C. one minute. A quality check was performed on the resulting library to ensure accurate loading on the Illumina® MiSeq.

The library was analyzed on the Invitrogen Qubit to determine concentration and then analyzed on the Fragment Analyzer (Advance Analytical, Ankeny, Iowa) using the Standard Sensitivity NGS Kit (DNF-473). Library molarity is calculated (ng/μl)*1500/average bp. The loading of this library was 4.6% of the MiSeq total pool. Standard loading is 2.3% of the total pool. The dual indexes in the adaptors were used to demultiplex the sequences and order these to the above sample and other samples on the same run.

The obtained paired read sequences were then demultiplexed a second time using flexbar version 2.5. First the R1 file with the forward reads was searched for the indexes, while keeping the according reverse reads attached. Then the R2 file with the reverse reads were searched for the indexes, while keeping the according forward reads attached. The following shell script was used:
#!/bin/bash
export PATH=/z/home/mcom/tools/flexbar_v2.5_linux64/:$PATH
export LD_LIBRARY_PATH=/z/home/mcom/tools/flexbar_v2.5_linux64:$LD_LIBRARY_PATH
B=B2RSJ # enter libary B### to analyze on
mkdir ${B}
cd ${B}
mkdir Flexbar
mkdir Flexbar/cat
mkdir Results
cd Flexbar
# we demultiplex on the Fw strand first and remove the barcode
flexbar -r ../../incoming/*${B}*_R1_001.fastq.gz -p ../../incoming/*${B}*_R2_001.fastq.gz -b ../../indexes/ELN-15-MCOM-0021_v2.fa -be LEFT -bt 0 -t ${B}_R1 -n 2
# we demultiplex next on the Rv strand and remove the barcode. The new file will have the old

Rv as the new Fw strand. hence, new rv strands contain wel barcode
flexbar -p ../../incoming/*${B}*_R1_001.fastq.gz -r ../../incoming/*${B}*_R2_001.fastq.gz -b ../../indexes/ELN-15-MCOM-0021_v2.fa -be LEFT -bt 0 -t ${B}_R2 -n 2
find -empty -type f -delete
echo $path
Is -l
for R in {A..A}
do
for C in {01..24}
do
cat *${B}*${R}${C}*_1.fastq>./cat/${B}${R}${C}_1.fastq
cat *${B}*${R}${C}*_2.fastq>./cat/${B}${R}${C}_2.fastq
done
done
cd cat
find -empty -type f -delete
echo $path
Is -l
cd ..#back to Flexbar
cd ..#back to B#
cd ..#back to NGSPlate
   A fasta file with the index sequences of table 11 below was used to demultiplex and sort the obtained Miseq sequences to A1 to A24:

TABLE 11

| Index | Sequence | SEQ ID NO: |
|---|---|---|
| >left_A01 | agacgtgcggtc | 90 |
| >left_A02 | tatgctgtggtc | 91 |
| >left_A03 | acatgtcgggtc | 92 |
| >left_A04 | ctatgcgcggtc | 93 |
| >left_A05 | atctatgcggtc | 94 |
| >left_A06 | agatgctaggtc | 95 |
| >left_A07 | tagatagcggtc | 96 |
| >left_A08 | atgtcagaggtc | 97 |
| >left_A09 | gcgtctgtccag | 98 |
| >left_A10 | ctatgtgtccag | 99 |
| >left_A11 | acgctgtcccag | 100 |
| >left_Al2 | atgtagcaccag | 101 |
| >left_A13 | gcagatgcgaag | 102 |
| >left_A14 | acgcgcgtgaag | 103 |
| >left_A15 | ctcgtatagaag | 104 |
| >left_A16 | tctactatgaag | 105 |
| >left_A17 | atagatgcctaa | 106 |
| >left_A18 | gatagagactaa | 107 |
| >left_A19 | atagtcgactaa | 108 |
| >left_A20 | gtcacgcgctaa | 109 |
| >left_A21 | actgactgccag | 110 |
| >left_A22 | agacgagtccag | 111 |
| >left_A23 | tcacacatccag | 112 |
| >left_A24 | cgatactgccag | 113 |
| >right_A01 | agacgtgcgttc | 114 |
| >right_A02 | tatgctgtgttc | 115 |
| >right_A03 | acatgtcggttc | 116 |
| >right_A04 | ctatgcgcgttc | 117 |
| >right_A05 | atctatgccaga | 118 |
| >right_A06 | agatgctacaga | 119 |
| >right_A07 | tagatagccaga | 120 |
| >right_A08 | atgtcagacaga | 121 |
| >right_A09 | gcgtctgtcaga | 122 |
| >right_A10 | ctatgtgtcaga | 123 |
| >right_A11 | acgctgtccaga | 124 |
| >right_Al2 | atgtagcacaga | 125 |
| >right_A13 | gcagatgccaga | 126 |
| >right_A14 | acgcgcgtcaga | 127 |
| >right_A15 | ctcgtatacaga | 128 |
| >right_A16 | tctactatcaga | 129 |
| >right_A17 | atagatgccaga | 130 |
| >right_A18 | gatagagacaga | 131 |
| >right_A19 | atagtcgacaga | 132 |
| >right_A20 | gtcacgcgcaga | 133 |
| >right_A21 | actgactggaag | 134 |
| >right_A22 | agacgagtgaag | 135 |
| >right_A23 | tcacacatgaag | 136 |
| >right_A24 | cgatactggaag | 137 |

The CLC Genomic work bench was used to map the sequences to the xylR-xylA-xylB references locus, get a voted consensus and a .tsv mapping coverage file. The coverage in the tsv ambiguity files was extracted for every tenth reference position using the following shell script using the library name as variable $1:
#!/bin/bash
echo “Reference name”$‘\t’“Reference position”$‘\t’“Reference sub-position (insertion)”$‘\t’“Reference symbol”$‘\t’“Number of A's”$‘\t’“Number of C's”$‘\t’“Number of G's”$‘\t’“Number of T's”$‘\t’“Number of N's”$‘\t’“Number of Gaps”$‘\t’“Total number of reads covering the position”>$1“_50_2260_coverage.tsv”
for P in {001..001}
do
for R in {A..A}
do
for C in {01..24}
do
for N in {50..2260..10}
do
LINE=$(grep -E “xylR xylA xylB”$‘\t’${N}$‘\t’ $1/Results/$1${R}${C}*.tsv)
echo $1“_”${R}${C}$‘\t’$LINE>>$1“_50_2260_coverage.tsv”
done
done
done
done

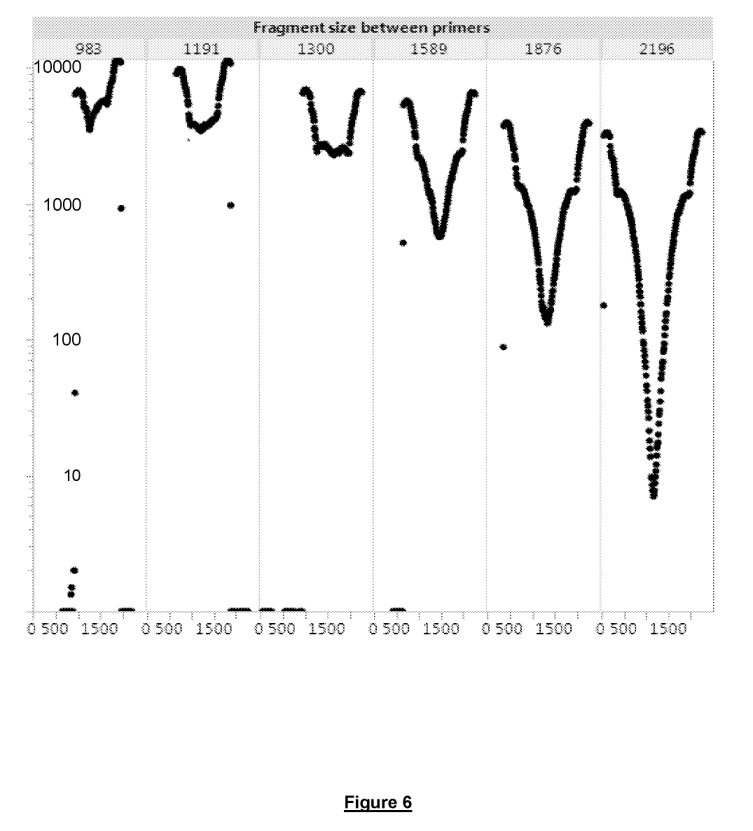

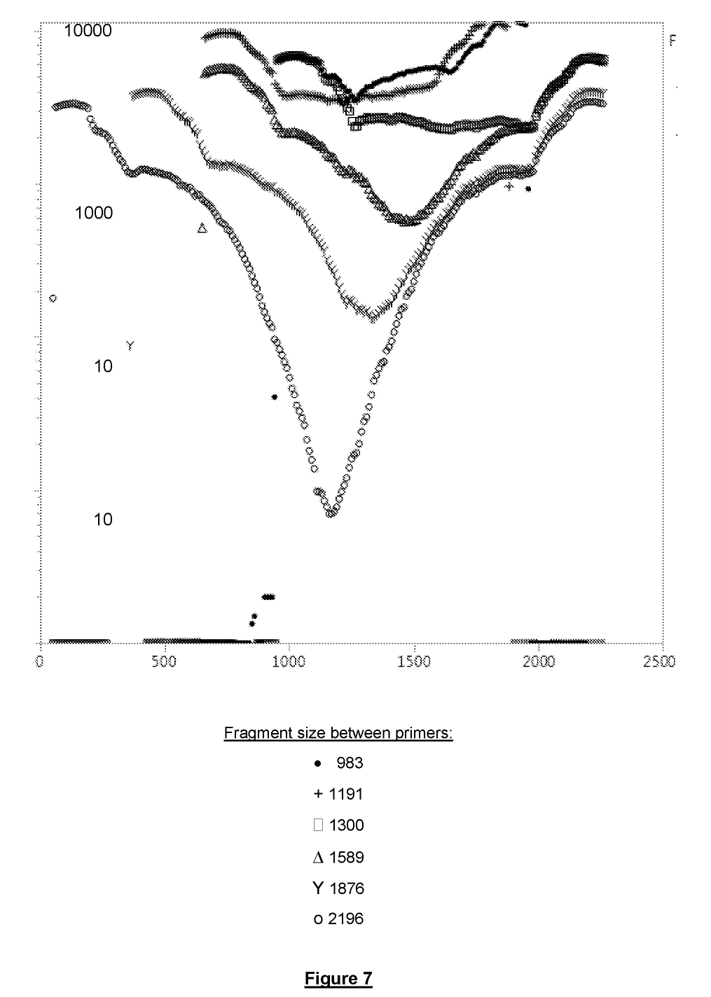

The Illumina® Miseq sequencing gave 1.533.750 total reads of which 311.732 and 299.106 contained index A1-24 in the forward and reverse reads, respectively. Hence, 40% of the total sequence contained the index. For each index the coverage at every tenth reference position was calculated. The average of the four replicates was plotted over the reference positions (see FIGS. 6 and 7).

This showed expectedly a higher coverage at the ends of the PCR amplicons which contained the index. The coverage decreased towards the middle of the PCR amplicon. Some points with low coverage outside the PCR amplicon were observed due to incomplete removal of the Illumina® TruSeq adaptors. These points are of no significance as they lie outside the sequence of interest.

The minimal coverage in the middle of the PCR amplicon decreased with increasing length of the amplicon. For a PCR amplicon size between the primers of 983 bp it was 3491×; for 1191 bp it was 3425×; for 1300 bp it was 2259×; for 1589 bp it was 557×; for 1876 bp it was 127× and for 2196 bp it was 6×. So, even with an amplicon of 2200 bp in size, sequencing coverage is obtained.

To compute the minimal coverage only positions from 1000 to 1800 of the xylR-xylA-xylB reference sequence were analysed as shown in Table 12.

TABLE 12
Statistics over coverage dependent on fragment size between the used primers and the used Primer set.
Primer set 1 to 4 for the 983 bp fragment is equivalent to the used naming A1-A4, and so on.

| Fragment | Mean over | | | | | | | | | |
|---|---|---|---|---|---|---|---|---|---|---|
| size | primer set 1 to 4 | | Mean coverage | | | | Minimum coverage | | | |
| between | Mean | Minimum | Primer | Primer | Primer | Primer | Primer | Primer | Primer | Primer |
| primers | coverage | coverage | set 1 | set 2 | set 3 | set 4 | set 1 | set 2 | set 3 | set 4 |
| 983 | 5.667 | 3.491 | 12.217 | 6.191 | 2.760 | 1.503 | 7.604 | 3.730 | 1725 | 905 |
| 1191 | 5.146 | 3.425 | 8.560 | 7.280 | 2.748 | 1.994 | 5.576 | 4.945 | 1872 | 1305 |
| 1300 | 3.435 | 2.259 | 5.860 | 5.372 | 1.478 | 1.029 | 4.026 | 3.334 | 999 | 678 |
| 1589 | 1.220 | 557 | 1.965 | 1.629 | 830 | 454 | 889 | 731 | 381 | 228 |
| 1876 | 462 | 127 | 718 | 699 | 288 | 141 | 201 | 214 | 61 | 32 |
| 2196 | 274 | 6 | 442 | 379 | 189 | 86 | 13 | 5 | 3 | 2 |

1. A method for determining the full coding sequences of a multitude of polynucleotides, said method comprising the steps of:
a) providing a multitude of polynucleotides;
b) adding at least one known oligonucleotide index to at least one end of each polynucleotide to produce elongated polynucleotides, wherein each known oligonucleotide index added to each polynucleotide is unique;
c) fragmenting the elongated polynucleotides;
d) sequencing the resulting fragments with paired forward and reverse reads using bridge amplification and next-generation sequencing;
e) sorting the paired forward and reverse sequence reads based on the at least one known oligonucleotide index; and
f) performing manual or in silico assembly of the sorted paired sequences, thereby providing the full coding sequence of each of the multitude of polynucleotides.

2. The method of claim 1, wherein the multitude of polynucleotides encodes one or more polypeptide of interest.

3. The method of claim 1, wherein the multitude of polynucleotides encodes variants of one or more polypeptide of interest.

4. The method of claim 1, wherein the multitude of polynucleotides comprises one or more promoters.

5. The method of claim 2, wherein the one or more polypeptide of interest is one or more enzyme, preferably selected from the group of enzymes consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

6. The method of claim 1, wherein the elongated polynucleotides in step (c) are randomly fragmented.

7. The method of claim 1, wherein the elongated polynucleotides in step (c) are fragmented by one or more endonuclease.

8. The method of claim 1, wherein the sorting step is done in silico.

9. The method of claim 1, wherein performing the assembly is aided by alignment with a known reference nucleotide sequence.
